(19) United States
(12) Patent Application Publication
(10) Pub. No.: US 2022/0081482 A1
(43) Pub. Date: Mar. 17, 2022

(54) ANTICANCER COMPOSITIONS COMPRISING IMMUNE CHECKPOINT INHIBITORS

(71) Applicant: NOAHM INC., Incheon (KR)

(72) Inventors: Man-Chul Suh, Incheon (KR); Yieun Jung, Gyeonggi-do (KR); Eun Ji Kim, Gangwon-do (KR); Jae In Jung, Gangwon-do (KR)

(21) Appl. No.: 17/419,634

(22) PCT Filed: Dec. 30, 2019
(86) PCT No.: PCT/KR2019/018697

§ 371 Date: Jun. 29, 2021

(30) Foreign Application Priority Data
Dec. 31, 2018 (KR) 10-2018-0173504

(51) Int. Cl. C07K 16/28; A61K 31/155; A61K 31/6615; A61K 31/7004; A61P 35/00

(52) U.S. Cl. CPC C07K 16/2818; C07K 16/2827; A61P 35/00; A61K 31/6615; A61K 31/7004; A61K 31/155

(57) ABSTRACT

The present invention relates to a pharmaceutical composition for preventing or treating cancer comprising: as active ingredients, (1) an immune checkpoint inhibitor; (2) a biguanide-based compound or a pharmaceutically acceptable salt thereof; (3) 2-deoxy-D-glucose; or further comprising: (4) inositol hexaphosphate or a pharmaceutically acceptable salt thereof, inositol, or a mixture thereof.

The compositions according to the present invention not only increase the indicants to various carcinomas for immune checkpoint inhibitors with limited indicants, but also exhibit a synergistic anti-cancer effect by appropriately combining specific drugs, thereby maximizing a therapeutic effect and killing only cancer cells without side effects. Therefore, the compositions of the present invention may be usefully used as anticancer agents for preventing or treating cancer.

### TECHNICAL FIELD

The present invention relates to an anticancer composition comprising an active ingredient capable of exhibiting an additional anticancer effect together with an immune checkpoint inhibitor.

### BACKGROUND ART

Cancer is one of representative intractable diseases that have been not conquered in modern medicine. In traditional methods of surgery, radiation, chemotherapy, etc., a short-term treatment effect may be exhibited, but in order to solve problems of many side effects such as cytotoxicity, metastasis, and relapse, the development of new therapeutic agents is urgently needed. The traditional tumor treatment methods have led to the development of targeted therapeutic agents through surgical procedures, chemical drugs, and recently, the importance of immunotherapy is greatly highlighted with the advent of immune checkpoint antibodies that regulate the immune environment (Couzin-Frankel, Science 2013; 342: 1432-1433).

Inhibition of the anticancer immune response has emerged as an important mechanism of tumor resistance to treatment, and through development of monoclonal antibodies that block cytotoxic T lymphocyte-associated antigen-4 (CTLA-4) and programmed death-1 (PD-1) or its ligand PD-L1 (programmed death-ligand 1) as immune checkpoint receptors, it is possible to stimulate and/or augment the endogenous anticancer immune response of patients.

This pathway may play a distinct role in immune regulation, and for example, CTLA-4 primarily regulates T cell proliferation in lymph nodes, while PD-1 mainly inhibits cells in a tumor microenvironment. These immune checkpoint inhibitors have recently verified excellent efficacy against various types of tumors, especially melanoma and non-small cell lung cancer, and quickly became a standard treatment method for patients.

To date, FDA-approved immune checkpoint inhibitors include total 6 of Yervoy® (CTLA4 inhibitor, ingredient name of ipilimumab) and Opdivo® (PD-1 inhibitor, ingredient name of nivolumab) from US pharmaceutical company, Bristol Myers Squibb (BMS), Keytruda® (PD-1 Inhibitor, ingredient name of pembrolizumab) from US pharmaceutical company, Merck (MSD), Tecentriq® (PD-L1 Inhibitor, ingredient name of atezolizumab) from Swiss Pharmaceutical Company, Roche, Bavencio® (PD-L1 inhibitor, ingredient name of avelumab) co-developed by US pharmaceutical company, Pfizer and Merck in Germany, and Imfinzi® (PD-L1 inhibitor, ingredient name of durvalumab) from British pharmaceutical company, AstraZeneca.

However, immune checkpoint inhibitors generally have immune related side effects that harm the gastrointestinal tract, endocrine glands, skin and liver. Most of these side effects are known to be associated with adverse response of an immune system as a result of activated T lymphocytes.

In addition, the immune checkpoint inhibitors are effective only in a small number of patients. In most advanced cancers, a response rate of anti-PD-1/PD-L1 monotherapy is about 20%, and a response rate of anti-CTLA-4 is about 12%, and thus it can be seen that there is a need for improvement (Borghaei et al., N Engl J Med. 2015; 373: 1627-1639). This low efficacy may be due to a lack of existing tumor-related T cell immunity (Elizabeth et al., Am J Clin Oncol. 2016 February; 39(1): 98-106).

In order to maximize the therapeutic effect of these immune checkpoint inhibitors, a clinical study combined with other anticancer drugs has been conducted. For example, two immune anticancer drugs (combination therapy of nivolumab and ipilimumab) for treating advanced melanoma were shown to have a greater effect of improving cell survival rate than each monotherapy. However, 4 of 10 persons had high side effects related to treatment enough to interrupt treatment. Using two or more treatment methods in combination when cancer was induced has been the cornerstone of cancer treatment today, but immune checkpoint inhibitors show a high response rate and a relatively high treatment interruption rate.

Therefore, research to increase the therapeutic effect while minimizing the side effects caused by anticancer drugs is urgently needed.

In addition, a main reason for the failure of chemotherapy is that the anticancer drug is effective initially, but gradually, drug resistance is expressed, and the immunity is extremely deteriorated. Therefore, there is a need for a method for improving the efficacy of cancer treatment without increasing the toxicity of the drug. The combination of anticancer drugs may be used as one method for improving the efficacy of the anticancer drugs, but unfortunately, combining anticancer drugs cannot all be expected to be synergistic, and finding a combination of drugs that have a synergistic effect is very difficult. Therefore, it is urgent to develop anticancer complex preparations that may maximize the anticancer effect while minimizing the side effects of anticancer drugs.

Recently, there has been much interest in developing anti-cancer therapies targeting cellular signal delivery pathways that are important for the metabolism and growth of cancer cells and representative drugs involving in the metabolism of the cancer cells includes metformin and 2-deoxy-D-glucose which are biguanide-based compounds (Wokoun et al., Oncol Rep. 2017; 37:2418-2424).

As an antihyperglycemic agent, metformin has been used as a first therapeutic agent for type 2 diabetes for decades. Despite the widespread use of metformin as an antidiabetic agent, potential anticancer effects in mammals were first reported in 2001. In addition, the first report on reducing cancer risk in patients with type 2 diabetes treated with metformin was published just 10 years ago. Since then, in many papers, metformin has shown consistent antiproliferative activity in several cancer cell lines including ovarian cancer, and xenotransplanted animals or transgenic mice. Regarding metabolism, metformin has been found as a new class of complex I and ATP synthase inhibitors, acts directly on mitochondria to restrict respiration and make energy inefficient and reduces glucose metabolism through citric acid circulation (Andrzejewski et al., 2014; 2: 12-25).

2-deoxy-D-glucose has been considered as a potential anticancer agent because of its dependence on tumor cells for glycolysis. 2-deoxy-D-glucose is a glucose analogue that can be easily absorbed by glucose transporters and acts as a competitive inhibitor of glycolysis, thereby reducing ATP production to induce cell death through activation of caspase-3 in solid tumors (Zhang et al., Cancer Lett. 2014; 355:176-183).

However, commonly used doses of metformin and 2-deoxy-D-glucose are insufficient to cure cancer sufficiently, and have a limitation in that adverse reactions may occur at high-dose treatment (Raez et al., Cancer Chemother Pharmacol. 2013; 71: 523-530). The combination treatment of metformin and 2-deoxy-D-glucose studied by Cheong et al. has been effective in breast cancer cell lines, but was higher than a concentration endurable in the human body or a concentration administrable in plasma (Cheong et al., Mol Cancer Ther. 2011; 10:2350-2362). In another paper, a combination of metformin and 2-deoxy-D-glucose has been successfully tested in prostate cancer cells using a metformin concentration higher than a concentration available in human plasma (Ben Sahra et al., Cancer Res. 2010; 70:2465-2475). This suggests that it is preferred that clinically effective anticancer agents, metformin and 2-deoxy-D-glucose lower therapeutic concentrations thereof within a range that can be reasonably achieved in vivo.

Meanwhile, inositol hexaphosphate and inositol are naturally organic phosphorous compounds which are contained in large amounts in most grains, seeds, and legumes and present even in mammalian cells, and are present together with a phosphate form (IP1-5) with low phosphates. Inositol hexaphosphate plays an important role in regulating important cellular functions such as signal transduction, cell proliferation and differentiation of various cells and is recognized as a natural antioxidant (Shamsuddin et al., J Nutr. 2003; 133:3778S-3784S).

Recently, it has been reported that inositol hexaphosphate had some effects of preventing cancer and inhibiting the growth, progression and metastasis of experimental tumors (Vucenik et al., Nutr Cancer. 2006; 55:109-125).

In preliminary clinical studies, it is reported that inositol hexaphosphate and inositol are administered in combination with chemotherapy to reduce the side effects of chemotherapy and improve the quality of life in patients having breast or colorectal cancer, suffered with liver metastasis. However, it is still difficult to effectively suppress cancer cell growth only by using inositol hexaphosphate or a combination formulation of inositol hexaphosphate and inositol.

### DISCLOSURE

### Technical Problem

Therefore, the present invention provides a pharmaceutical composition for preventing or treating cancer comprising: as active ingredients, (1) an immune checkpoint inhibitor; (2) a biguanide-based compound or a pharmaceutically acceptable salt thereof; and (3) 2-deoxy-D-glucose.

The present invention also provides a pharmaceutical composition for preventing or treating cancer comprising: as active ingredients, (1) an immune checkpoint inhibitor; (2) a biguanide-based compound or a pharmaceutically acceptable salt thereof; (3) 2-deoxy-D-glucose; and (4) inositol hexaphosphate or a pharmaceutically acceptable salt thereof, inositol, or a mixture thereof.

The pharmaceutical composition may effectively kill cancer cells even with a combination of each compounds at a low concentration, and is expected to be widely used in the field of cancer treatment in the future as well as to secure the safety of the human body, and particularly, exhibits a cancer cell-specific toxic effect to serve as a pharmaceutical composition for preventing or treating cancer with reduced side effects.

### Technical Solution

As one aspect to achieve the objects, the present invention provides a pharmaceutical composition for preventing or treating cancer comprising: as active ingredients, (1) an immune checkpoint inhibitor; (2) a biguanide-based compound or a pharmaceutically acceptable salt thereof; and (3) 2-deoxy-D-glucose.

As another aspect to achieve the objects, the present invention provides a pharmaceutical composition for preventing or treating cancer comprising: (1) an immune checkpoint inhibitor; (2) a biguanide-based compound or a pharmaceutically acceptable salt thereof; and (3) 2-deoxy-D-glucose

In addition, the pharmaceutical composition for preventing or treating cancer may further comprise (4) inositol hexaphosphate or a pharmaceutically acceptable salt thereof, inositol, or a mixture thereof as an additional active ingredient.

The present invention also provides a pharmaceutical composition for preventing or treating cancer comprising: as active ingredients, (1) an immune checkpoint inhibitor; (2) a biguanide-based compound or a pharmaceutically acceptable salt thereof; (3) 2-deoxy-D-glucose; and (4) inositol hexaphosphate or a pharmaceutically acceptable salt thereof, inositol, or a mixture thereof.

The present invention also provides a pharmaceutical composition for preventing or treating cancer comprising: (1) an immune checkpoint inhibitor; (2) a biguanide-based compound or a pharmaceutically acceptable salt thereof; (3) 2-deoxy-D-glucose; and (4) inositol hexaphosphate or a pharmaceutically acceptable salt thereof, inositol, or a mixture thereof.

The pharmaceutical compositions according to the present invention include a small amount of active ingredients to have an excellent effect that can effectively treat cancer with fewer side effects. The pharmaceutical compositions according to the present invention may use a smaller amount of individual compound included in the complex formulation than when treated with a single compound, thereby significantly reducing the risk and/or severity of side effects and significantly increasing the overall effect of the treatment.

In particular, according to the present invention, immune checkpoint inhibitors that block CTLA-4 and PD-1 or its ligand PD-L1, which are immune checkpoint receptors, have an excellent anticancer effect even at low concentrations when included and used in the pharmaceutical composition according to the present invention.

Hereinafter, the present invention will be described in detail.

The pharmaceutical composition of the present invention comprises (1) an immune checkpoint inhibitor as an active ingredient.

The immune checkpoint inhibitors may treat cancer by inhibiting immune evasion of cancer by blocking immune check points that prevent the progress of an immune response in cancers with high immune inhibition ability. The immune checkpoint inhibitors are new tumor therapeutic agents that have been developed as a result of much understanding of the human immune system due to development of immunology and have been widely used in anticancer strategies. As an exemplary mechanism for exerting anticancer effects using immune checkpoint inhibitors, there are a T lymphocyte inhibition mechanism by CTLA-4 and a PD-1/PD-L1 mechanism for inhibiting pre-activated T lymphocytes. However, it has been reported that there is a limitation in that treatment using only an immune checkpoint inhibitor has low treatment efficiency and a slight effect.

However, the pharmaceutical composition of the present invention helps in preventing and treating cancer due to a synergistic complementary effect by administering active ingredients of another therapeutic mechanism in combination with an immune checkpoint inhibitor.

The immune checkpoint inhibitor may be an antibody, a fusion protein, an aptamer or an immune checkpoint protein-binding fragment thereof. For example, an immune checkpoint inhibitor is an anti-immune checkpoint protein antibody or an antigen-binding fragment thereof.

In a specific example, the immune checkpoint inhibitor is at least one selected from the group consisting of an anti-CTLA4 antibody, a derivative thereof or an antigen-binding fragment thereof; an anti-PD-L1 antibody, a derivative thereof or an antigen-binding fragment thereof; an anti-LAG-3 antibody, a derivative thereof or an antigen-binding fragment thereof; an anti-OX40 antibody, a derivative thereof or an antigen-binding fragment thereof; an anti-TIM3 antibody, a derivative thereof or an antigen-binding fragment thereof; and an anti-PD-1 antibody, a derivative thereof or an antigen-binding fragment thereof.

For example, the immune checkpoint inhibitor may be at least one selected from the group consisting of ipilimumab, a derivative thereof or an antigen-binding fragment thereof; tremelimumab, a derivative thereof or an antigen-binding fragment thereof; nivolumab, a derivative thereof or an antigen-binding fragment thereof; pembrolizumab, a derivative thereof or an antigen-binding fragment thereof; pidilizumab, a derivative thereof or an antigen-binding fragment thereof; atezolizumab, a derivative thereof or an antigen-binding fragment thereof; durvalumab, a derivative thereof or an antigen-binding fragment thereof; avelumab, a derivative thereof or an antigen-binding fragment thereof; BMS-936559, a derivative thereof or an antigen-binding fragment thereof; BMS-986016, a derivative thereof or an antigen-binding fragment thereof; GSK3174998, a derivative thereof or an antigen-binding fragment thereof; TSR-022, a derivative thereof or an antigen-binding fragment thereof; MB G453, a derivative thereof or an antigen-binding fragment thereof; LY3321367, a derivative thereof or an antigen-binding fragment thereof; and an IMP321 recombinant fusion protein. Any antibody that can be used as an immune checkpoint inhibitor or other types of immune checkpoint inhibitors may be used without limitation.

Specifically, the immune checkpoint inhibitor may be preferably at least one selected from the group consisting of an anti-CTLA4 antibody, an anti-PD-1 antibody, an anti-LAG-3 antibody, an anti-OX40 antibody, an anti-TIM3 antibody, and an anti-PD-L1 antibody. The antibody may be purchased and used, for example, from a conventional antibody manufacturer and the like, or may be prepared and used according to a known antibody production method.

The immune checkpoint inhibitor may be a small molecular compound that has an effect as the above-mentioned immune checkpoint inhibitor or is involved in an inhibitory mechanism thereof. Such small molecular compounds can be, for example, small molecule compounds that bind to an immune checkpoint protein or are involved in mechanisms involved in immune checkpoint inhibition.

Specifically, the small molecular compounds may be BMS-202 (Source: BMS), BMS-8 (Source: BMS), CA170 (Source: Curis/Aurigene), CA327 (Source: Curis/Aurigene), Epacadostat, GDC-0919, BMS-986205, and the like. Any small molecular compounds that are used as an immune checkpoint inhibitor or has a related effect may be used without limitation.

The pharmaceutical composition of the present invention comprises (2) a biguanide-based compound or a pharmaceutically acceptable salt thereof as an active ingredient.

In the present invention, the biguanide-based compound is, for example, metformin or phenformin. Specifically, metformin has a structural formula of Chemical Formula 1.

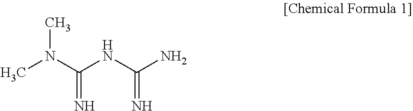

Specifically, phenformin has a structural formula of Chemical Formula 2.

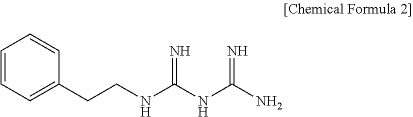 [SMILES: N=C(N)NC(=N)NCCc1ccccc1]

In the present invention, when (1) an immune checkpoint inhibitor; (2) a biguanide-based compound or a pharmaceutically acceptable salt thereof; and (3) 2-deoxy-D-glucose are used as a complex formulation, the compounds exhibit a high anticancer effect even at a low concentration. In addition, it shows a better anticancer effect when it further comprises (4) inositol hexaphosphate or a pharmaceutically acceptable salt thereof, inositol, or a mixture thereof.

The biguanide-based drugs are not limited thereto, but can have an anticancer effect through an action mechanism that activates an enzyme called AMP-activated kinase (AMPK), which plays a pivotal role in intracellular energy balance and nutrient metabolic regulation.

When metformin is orally administered to rats, it can be seen that metformin, LD50 thereof is 1,450 mg/kg, is a very safe compound, but there is still a problem that metformin needs to be used in high doses. Meanwhile, phenformin was developed in the late 1950s as an oral diabetes treatment, and was intended to be used for the treatment of insulin-independent diabetes (type 2 diabetes), but due to a serious side effect called lactic acidosis, the use of phenformin was completely banned in the late 1970s.

It was confirmed that the complex formulation above-mentioned exhibited a high anticancer effect even at a much lower concentration than that of each single agent or a composition of a combination of two compounds, thereby improving a problem of high-dose administration or a problem of side effects of metformin or phenformin

The pharmaceutical composition of the present invention comprises (3) 2-deoxy-D-glucose as an active ingredient.

In the present invention, 2-deoxy-D-glucose has a structure represented by the Chemical Formula 3.

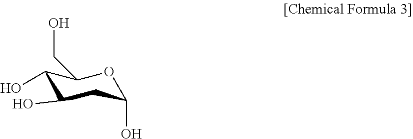

The compound of Chemical Formula 3 has an action effect as an inhibitor of glycolysis. 2-deoxy-D-glucose, a derivative of glucose, has an action effect of inhibiting glycolysis in a glucose metabolism and inhibiting glycosylation of proteins in the endoplasmic reticulum to induce vesicle stress. As such, 2-deoxy-D-glucose, an inhibitor of glucose degradation, has not been shown to kill cancer cells by itself, but forms a complex formulation of the present invention to have excellent anticancer effects.

The pharmaceutical composition of the present invention may further comprise (4) inositol hexaphosphate or a pharmaceutically acceptable salt thereof, inositol, or a mixture thereof as an additional active ingredient.

In the present invention, inositol hexaphosphate and/or inositol may regulate several important pathways in cancer cells. In the present invention, inositol hexaphosphate specifically has a structure of Chemical Formula 4.

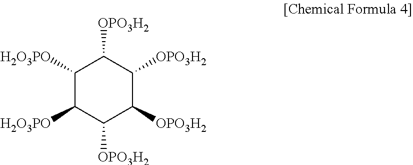

In the present invention, inositol specifically has a structure of Chemical Formula 5.

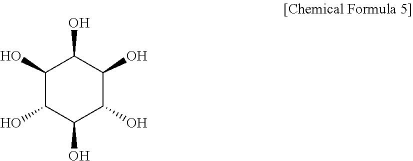 [SMILES: O[C@H]1[C@H](O)[C@@H](O)[C@H](O)[C@@H](O)[C@H]1O]

In the present invention, it was confirmed that inositol hexaphosphate or a pharmaceutically acceptable salt thereof, inositol, or a mixture thereof are combined with an immune checkpoint inhibitor, a biguanide-based compound or a pharmaceutically acceptable salt thereof, and 2-deoxy-D-glucose to have a high anticancer effect even at a low concentration.

In the present invention, the biguanide-based compound and inositol hexaphosphate may be present in the form of a pharmaceutically acceptable salt. As the salt, acid addition salts formed with pharmaceutically acceptable free acids are useful. The term “pharmaceutically acceptable salt” used in the present invention refers to any organic or inorganic addition salt in which at a concentration having relatively non-toxic and harmless effects on a patient, side effects caused by the salt does not degrade a beneficial effect of the biguanide-based compound and inositol hexaphosphate.

At this time, as the addition salt, hydrochloric acid, phosphoric acid, sulfuric acid, nitric acid, tartaric acid, etc. may be used as inorganic acid, and methanesulfonic acid, p-toluenesulfonic acid, acetic acid, trifluoroacetic acid, maleic acid, succinic acid, oxalic acid, benzoic acid, tartaric acid, fumaric acid, manderic acid, propionic acid, citric acid, lactic acid, glycolic acid, gluconic acid, galacturonic acid, glutamic acid, glutaric acid, glucuronic acid, aspartic acid, ascorbic acid, carbonic acid, vanillic acid, hydroiodic acid, etc. may be used as organic acid, but are not limited thereto.

Further, bases may also be used to prepare pharmaceutically acceptable metal salts. An alkali metal salt or an alkaline earth metal salt may be obtained, for example, by dissolving the compound in a large amount of alkali metal hydroxide or alkaline earth metal hydroxide solution, filtering a non-dissolved compound salt, and then evaporating and drying a filtrate. In this case, the metal salt is pharmaceutically suitable to prepare, particularly, sodium, potassium, calcium, and magnesium salts or mixed salts thereof, but is not limited thereto.

A pharmaceutically acceptable salt of each of the biguanide-based compound (metformin or phenformin) and inositol hexaphosphate includes a salt of acid or basic group which may be present in each of the biguanide-based compound (metformin or phenformin) and inositol hexaphosphate, unless otherwise indicated. For example, the pharmaceutically acceptable salt may include sodium, potassium, calcium or magnesium salts and the like of a hydroxy group, and other pharmaceutically acceptable salts of an amino group include hydrobromide, sulfate, hydrogen sulfate, phosphate, hydrogen phosphate, dihydrogen phosphate, acetate, succinate, citrate, tartrate, lactate, mandelate, methanesulfonate (mesylate), and p-toluenesulfonate (tosylate) salts and the like, which may be prepared through a method for preparing a salt known in the art.

As the salt of the biguanide-based compound (metformin or phenformin) of the present invention, as a pharmaceutically acceptable salt, any of metformin or phenformin salts exhibiting anticancer effects equivalent to metformin or phenformin may be used. Preferably, metformin hydrochloride, metformin succinate, metformin citrate or phenformin hydrochloride, phenformin succinate, phenformin citric acid and the like may be used, but are not limited thereto.

As the inositol hexaphosphate salt of the present invention, as a pharmaceutically acceptable salt, any inositol hexaphosphate salt exhibiting an anticancer effect equivalent to inositol hexaphosphate may be used. Preferably, inositol hexaphosphate sodium, inositol hexaphosphate potassium, inositol Hexaphosphate calcium, inositol hexaphosphate ammonium, inositol hexaphosphate magnesium, inositol hexaphosphate calcium magnesium, and the like may be used, but are not limited thereto.

The biguanide-based compound, 2-deoxy-D-glucose, and inositol hexaphosphate of the present invention also include derivatives thereof. The term “derivative” refers to a compound prepared by chemically changing a part of the compound, for example, introduction, substitution, and deletion of a functional group, so long as the anticancer activity of the compound is not changed, and it can be included without limitation in the present invention.

In the present invention, inositol may exist in the form of various isomers. The isomers include both enantiomers and diastereomers. Any inositol that has a pharmacologically anticancer effect all may be used, and preferably, at least one selected from the group consisting of D-chiro-inositol, L-chiro-inositol, myo-inositol, and scyllo-inositol may be used, but is not limited thereto.

Even though combining two or more drugs, each of which is known to have an anticancer effect, it cannot be expected that the combined drug exhibits a synergistic effect, and rather, a function of the drug is offset by the combination, so that it is very difficult to find a combination of drugs having a synergistic effect. In the present invention, an anticancer complex formulation capable of maximizing the anticancer effect while minimizing side effects by using a minimum concentration of anticancer drugs has been developed.

In the present invention, preferred aspect of pharmaceutical composition for preventing or treating cancer may be
a composition comprising an immune checkpoint inhibitor, a biguanide-based compound or a pharmaceutically acceptable salt thereof, and 2-deoxy-D-glucose;
a composition comprising an immune checkpoint inhibitor, a biguanide-based compound or a pharmaceutically acceptable salt thereof, 2-deoxy-D-glucose, and inositol hexaphosphate or a pharmaceutically acceptable salt thereof;
a composition comprising an immune checkpoint inhibitor, a biguanide-based compound or a pharmaceutically acceptable salt thereof, 2-deoxy-D-glucose, and inositol; or a composition comprising an immune checkpoint inhibitor, a biguanide-based compound or a pharmaceutically acceptable salt thereof, 2-deoxy-D-glucose, inositol hexaphosphate or a pharmaceutically acceptable salt thereof and inositolln the present invention, the immune checkpoint inhibitor may be at least one selected from the group consisting of, for example, an anti-CTLA4 antibody, an anti-PD-1 antibody, an anti-LAG-3 antibody, an anti-OX40 antibody, an anti-TIM3 antibody, an anti-PD-1 antibody and an anti-PD-L1 antibody. More preferably, the immune checkpoint inhibitor is any one selected from the group consisting of an anti-CTLA4 antibody, an anti-PD-1 antibody and an anti-PD-L1 antibody. Alternatively, the immune checkpoint inhibitor may be a small molecular compound that binds to an immune checkpoint protein or is involved in a mechanism involved in immune checkpoint inhibition.

For example, as one aspect for achieving the object, a complex formulation of anti-CTLA-4 antibody/metformin/2-deoxy-D-glucose, a complex formulation of anti-CTLA-4 antibody/metformin/2-deoxy-D-glucose/inositol hexaphosphate, a complex formulation of anti-CTLA-4 antibody/metformin/2-deoxy-D-glucose/inositol, and a complex formulation of anti-CTLA-4 antibody/metformin/2-deoxy-D-glucose/inositol hexaphosphate/inositol are much more effective in reducing the tumor size than a single formulation of each compound or a complex formulation included two compounds.

Further, as another aspect for achieving the object, a complex formulation of anti-PD-1 antibody/metformin/2-deoxy-D-glucose, a complex formulation of anti-PD-1 antibody/metformin/2-deoxy-D-glucose/inositol hexaphosphate, a complex formulation of anti-PD-1 antibody/metformin/2-deoxy-D-glucose/inositol, and a complex formulation of anti-PD-1 antibody/metformin/2-deoxy-D-glucose/inositol hexaphosphate/inositol are much more effective in reducing the tumor size than a single formulation of each compound or a complex formulation included two compounds.

Further, as another aspect for achieving the object, a complex formulation of anti-PD-L1 antibody/metformin/2-deoxy-D-glucose, a complex formulation of anti-PD-L1 antibody/metformin/2-deoxy-D-glucose/inositol hexaphosphate, a complex formulation of anti-PD-L1 antibody/metformin/2-deoxy-D-glucose/inositol, and a complex formulation of anti-PD-L1 antibody/metformin/2-deoxy-D-glucose/inositol hexaphosphate/inositol are much more effective in reducing the tumor size than a single formulation of each compound or a complex formulation included two compounds

In the present invention, the biguanide-based compound may be metformin or a pharmaceutically acceptable salt thereof, or phenformin or a pharmaceutically acceptable salt thereof.

When describing a composition centered on metformin as the biguanide-based compound, the pharmaceutical composition for preventing or treating cancer of the present invention may be
a composition comprising an immune checkpoint inhibitor, metformin or a pharmaceutically acceptable salt thereof, and 2-deoxy-D-glucose;
a composition comprising an immune checkpoint inhibitor, metformin or a pharmaceutically acceptable salt thereof, 2-deoxy-D-glucose, and inositol hexaphosphate or a pharmaceutically acceptable salt thereof;
a composition comprising an immune checkpoint inhibitor, metformin or a pharmaceutically acceptable salt thereof, 2-deoxy-D-glucose, and inositol; or
a composition comprising an immune checkpoint inhibitor, metformin or a pharmaceutically acceptable salt thereof, 2-deoxy-D-glucose, inositol hexaphosphate or a pharmaceutically acceptable salt thereof and inositol.

When describing a composition centered on phenformin as another biguanide-based compound, the composition for preventing or treating cancer of the present invention may be
a composition comprising an immune checkpoint inhibitor, phenformin or a pharmaceutically acceptable salt thereof, and 2-deoxy-D-glucose;
a composition comprising an immune checkpoint inhibitor, phenformin or a pharmaceutically acceptable salt thereof, 2-deoxy-D-glucose, and inositol hexaphosphate or a pharmaceutically acceptable salt thereof;
a composition comprising an immune checkpoint inhibitor, phenformin or a pharmaceutically acceptable salt thereof, 2-deoxy-D-glucose, and inositol; or
a composition comprising an immune checkpoint inhibitor, phenformin or a pharmaceutically acceptable salt thereof, 2-deoxy-D-glucose, inositol hexaphosphate or a pharmaceutically acceptable salt thereof and inositol. In one embodiment of the present invention, it is confirmed that the pharmaceutical composition according to the present invention may inhibit cancer with a remarkably excellent effect in a cancer animal model. That is, according to the present invention, each of immune checkpoint inhibitor, a biguanide-based compound, 2-deoxy-D-glucose, inositol hexaphosphate and inositol alone, a large amount thereof needs to be used due to insufficient anti-cancer effects, but when a complex formulation combining the compounds is used, it was confirmed that cancer cells may be effectively killed even in a small amount.

In each pharmaceutical composition according to the present invention, a weight ratio of the combination of each compound is not particularly limited.

For example, as the immune checkpoint inhibitor, single dosage of at least one selected from the group consisting of monoclonal antibodies, for example, an anti-CTLA4 antibody, an anti-PD-1 antibody, an anti-LAG-3 antibody, an anti-OX40 antibody, an anti-TIM3 antibody, an anti-PD-1 antibody and an anti-PD-L1 antibody may be used in a range of 0.01 to 25 mg/kg.

The compounds other than immune checkpoint inhibitor may be used in the following ranges, depending on a type of cancer to be treated.

For example, a weight ratio of metformin or a pharmaceutically acceptable salt thereof: 2-deoxy-D-glucose may be a range of 1:0.2 to 1:5, and a relative amount of combination may vary depending on a type of cancer to be treated.

For example, a weight ratio of phenformin or a pharmaceutically acceptable salt thereof: 2-deoxy-D-glucose may be a range of 1:1 to 1:50, and a relative amount of combination may vary depending on a type of cancer to be treated.

In another example, a weight ratio of metformin or a pharmaceutically acceptable salt thereof: 2-deoxy-D-glucose: inositol hexaphosphate or a pharmaceutically acceptable salt thereof may be a range of 1:0.2:0.5 to 1:5:20, and a relative amount of combination may vary depending on a type of cancer to be treated.

In another example, a weight ratio of phenformin or a pharmaceutically acceptable salt thereof: 2-deoxy-D-glucose: inositol hexaphosphate or a pharmaceutically acceptable salt thereof may be a range of 1:1:1 to 1:50:200, and a relative amount of combination may vary depending on a type of cancer to be treated.

In another example, a weight ratio of metformin or a pharmaceutically acceptable salt thereof: 2-deoxy-D-glucose: inositol may be a range of 1:0.2:0.5 to 1:5:20, and a relative amount of combination may vary depending on a type of cancer to be treated.

In another example, a weight ratio of phenformin or a pharmaceutically acceptable salt thereof: 2-deoxy-D-glucose: inositol may be a range of 1:1:1 to 1:50:200, and a relative amount of combination may vary depending on a type of cancer to be treated.

In another example, a weight ratio of metformin or a pharmaceutically acceptable salt thereof: 2-deoxy-D-glucose: inositol hexaphosphate or a pharmaceutically acceptable salt thereof: inositol may be a range of 1:0.2:0.5:0.5 to 1:5:20:20, and a relative amount of combination may vary depending on a type of cancer to be treated.

In another example, a weight ratio of phenformin or a pharmaceutically acceptable salt thereof: 2-deoxy-D-glucose: inositol hexaphosphate or a pharmaceutically acceptable salt thereof: inositol may be a range of 1:1:1:1 to 1:50:200:200, and a relative amount of combination may vary depending on a type of cancer to be treated. The present invention also provides a pharmaceutical composition for preventing or treating cancer comprising: as active ingredients, (1) an immune checkpoint inhibitor; (2) a biguanide-based compound or a pharmaceutically acceptable salt thereof; (3) 2-deoxy-D-glucose; and (4) inositol hexaphosphate or a pharmaceutically acceptable salt thereof, inositol, or a mixture thereof. The definitions of (1) to (4) are the same as those described above.

The term “cancer” used in the present invention refers to a disease associated with cell death control, and refers to a disease caused by excessive proliferation of cells when a normal apoptotic balance is broken. These abnormally overproliferating cells invade surrounding tissues and organs to form masses in some cases and destroy or modify the normal structure of the body, which is called cancer. In general, the term “tumor” refers to a mass grown abnormally by autonomous overgrowth of body tissues, and may be classified into a benign tumor and a malignant tumor. The malignant tumor grows much faster than benign tumor, and invades surrounding tissues, as a result, metastasis occurs to threaten the life. The malignant tumor is commonly referred to as ‘cancer’, and the types of cancer include cerebral spinal cord tumor, brain cancer, head and neck cancer, lung cancer, breast cancer, thymic tumor, esophageal cancer, stomach cancer, colon cancer, liver cancer, pancreatic cancer, biliary tract cancer, kidney cancer, bladder cancer, prostate cancer, testicular cancer, germ cell tumor, ovarian cancer, cervical cancer, endometrial cancer, lymphoma, leukemia such as acute leukemia or chronic leukemia, osteosarcoma, multiple myeloma, sarcoma, melanoma, malignant melanoma, and skin cancer, and the like. The anticancer composition of the present invention may be used without limitation to the type of cancer, but may be used for preventing or treating at least one selected from the group consisting of liver cancer, lung cancer, stomach cancer, pancreatic cancer, colon cancer, cervical cancer, breast cancer, prostate cancer, ovarian cancer, brain cancer, osteosarcoma, bladder cancer, head and neck cancer, kidney cancer, melanoma, leukemia and lymphoma.

The term “preventing or treating” used in the present invention refers to all actions that inhibit or delay the development of cancer using the pharmaceutical composition or complex formulation according to the present invention, and particularly, “treating” refers to all actions of improving or beneficially modifying cancer using the composition.

Therefore, the present invention provides a method for treating cancer comprising administering a therapeutically effective amount of (1) an immune checkpoint inhibitor; (2) a biguanide-based compound or a pharmaceutically acceptable salt thereof; and (3) 2-deoxy-D-glucose to a subject in need of treatment of cancer. The method may further comprise administering a therapeutically effective amount of (4) inositol hexaphosphate or a pharmaceutically acceptable salt thereof, inositol, or a mixture thereof.

The term “administering” used in the present invention refers to providing a subject with the pharmaceutical composition or complex formulation according to the present invention in any suitable manner. At this time, the subject refers to an animal, and may be a mammal that can exhibit a beneficial effect, typically with treatment with the pharmaceutical composition or complex formulation according to the present invention. Preferred examples of such individuals may include primates such as humans.

The pharmaceutical composition for preventing or treating cancer of the present invention may further include a chemotherapeutic agent for treating cancer, if necessary, in addition to the above-mentioned active ingredients.

In addition, the pharmaceutical composition for preventing or treating cancer of the present invention may further include a pharmaceutically acceptable carrier. The composition of the present invention, according to the purpose of use, may be formulated and used by oral formulation such as powders, granules, tablets, capsules, suspensions, emulsions, syrups, and aerosols and the like, sterile injectable solutions, external forms such as ointments and the like, and suppositories and the like according to general methods. A carrier, an excipient, and a diluent which may be included in such composition may include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and, mineral oil and the like.

A solid formulation for oral administration includes a tablet, a pill, a powder, a granule, a capsule, and the like, and such solid formulation may be formulated by mixing at least one excipient, for example, starch, calcium carbonate, sucrose, lactose, gelatin, and the like with the composition. Further, lubricants such as magnesium stearate and talc may also be used in addition to simple excipients. A liquid formulation for oral administration may correspond to a suspension, an internally applied solution, an emulsion, a syrup, and the like, and may include various excipients, for example, a wetting agent, a sweetener, an aromatic agent, a preserving agent, and the like in addition to water and liquid paraffin which are commonly used as simple diluents.

A formulation for parenteral administration includes a sterile aqueous solution, a non-aqueous solution, a suspension, an emulsion, and a lyophilizing agent, and a suppository. As the non-aqueous solution and the suspension, propylene glycol, polyethylene glycol, vegetable oil such as olive oil, injectable ester such as ethyl oleate, and the like may be used. Bases for the injectable agent may include conventional additives such as solubilizers, isotonic agents, suspending agents, emulsifiers, stabilizers and preservatives.

The composition of the present invention may be administered using a variety of methods, such as oral, intravenous, subcutaneous, intradermal, intranasal, intraperitoneal, intramuscular, and transdermal administration and the like, and the dosage may vary depending on the age, gender, and weight of the patient and may be easily determined by those skilled in the art. The dosage of the composition according to the present invention may be increased or decreased depending on a route of administration, the severity of the disease, gender, weight, age, and the like. For example, in the case of a complex formulation of five compounds, anti-PD-1, monoclonal antibody as the immune checkpoint inhibitor, may be used with 0.01 to 25 mg/kg (body weight) as single dosage, metformin as the biguanide-based compound may be used with 5 to 80 mg/kg (body weight) per day, phenformin may be used with 0.1 to 10 mg/kg (body weight) per day, 2-deoxy-D-glucose may be used with 0.1 to 160 mg/kg (body weight) per day, inositol hexaphosphate may be used with 2 to 600 mg/kg (body weight) per day, and inositol may be used with 2 to 600 mg/kg (body weight) per day. However, the scope of the present invention is not limited by the dosage.

As another aspect, the present invention relates to a method for treating cancer comprising administering, to a subject, (1) an immune checkpoint inhibitor; (2) a biguanide-based compound or a pharmaceutically acceptable salt thereof; and (3) 2-deoxy-D-glucose.

As another aspect, the present invention relates to a method for treating cancer comprising administering, to a subject, (1) an immune checkpoint inhibitor; (2) a biguanide-based compound or a pharmaceutically acceptable salt thereof; (3) 2-deoxy-D-glucose; and (4) inositol hexaphosphate or a pharmaceutically acceptable salt thereof, inositol, or a mixture thereof.

As yet another aspect, the present invention relates to a composition for use in the treatment of cancer comprising: (1) an immune checkpoint inhibitor; (2) a biguanide-based compound or a pharmaceutically acceptable salt thereof; and (3) 2-deoxy-D-glucose.

As yet another aspect, the present invention relates to a composition for use in the treatment of cancer comprising: (1) an immune checkpoint inhibitor; (2) a biguanide-based compound or a pharmaceutically acceptable salt thereof; (3) 2-deoxy-D-glucose; and (4) inositol hexaphosphate or a pharmaceutically acceptable salt thereof, inositol, or a mixture thereof.

As yet another aspect, the present invention relates to a use of composition comprising: (1) an immune checkpoint inhibitor; (2) a biguanide-based compound or a pharmaceutically acceptable salt thereof; and (3) 2-deoxy-D-glucose for the manufacture of a medicament for the prevention or treating cancer.

As yet another aspect, the present invention relates to a use of composition comprising: (1) an immune checkpoint inhibitor; (2) a biguanide-based compound or a pharmaceutically acceptable salt thereof; (3) 2-deoxy-D-glucose; and (4) inositol hexaphosphate or a pharmaceutically acceptable salt thereof, inositol, or a mixture thereof for the manufacture of a medicament for the prevention or treating cancer.

### Advantageous Effects

The compositions of the present invention not only increase the indicants to various carcinomas for immune checkpoint inhibitors with limited indicants, but also exhibit a synergistic anti-cancer effect by appropriately combining specific drugs, thereby maximizing a therapeutic effect and killing only cancer cells without side effects. Therefore, the compositions of the present invention may be usefully used as anticancer agents for preventing or treating cancer.

### DESCRIPTION OF DRAWINGS

FIGS. 1 and 2 are graphs showing cell survival rate by MTT assay as a percentage after 48 hours by treating single and complex formulations of metformin (MET), 2-deoxy-D-glucose (2DG) and inositol hexaphosphate (IP6) to human-derived cancer cell lines at a low concentration usable in human plasma. A vertical bar of each bar represents a standard deviation. Statistical analysis was performed by one-way ANOVA testing using Tukey's multiple comparison post analysis using GraphPad Prism 6.0 software.

FIG. 1A is a graph of examining a HepG2 cell line as cancer cells derived from human liver. FIG. 1B is a graph of examining an A549 cell line as cancer cells derived from human lung. FIG. 1C is a graph of examining an AGS cell line as cancer cells derived from human stomach. FIG. 1D is a graph of examining a PANC-1 cell line as cancer cells derived from human pancreas. FIG. 1E is a graph of examining a DLD-1 cell line as cancer cells derived from human colon. FIG. 1F is a graph of examining a HeLa cell line as cancer cells derived from human cervix. FIG. 2A is a graph of examining an MDA-MB-231 cell line as cancer cells derived from human breast. FIG. 2B is a graph of examining a PC-3 cell line as cancer cells derived from human prostate. FIG. 2C is a graph of examining an SK-OV-3 cell line as cancer cells derived from human ovary. FIG. 2D is a graph of examining a T24 cell line as cancer cells derived from human bladder. FIG. 2E is a graph of examining a U-87 MG cell line as cancer cells derived from human brain. FIG. 2F is a graph of examining a Saos-2 cell line as cancer cells derived from human bones. ****p<0.0001.

FIGS. 3 and 4 are graphs showing cell survival rate by MTT assay as a percentage after 48 hours by treating single and complex formulations of phenformin (PHE), 2DG, and IP6 to human-derived cancer cell lines at a low concentration usable in human plasma. A vertical bar of each bar represents a standard deviation. Statistical analysis was performed by one-way ANOVA testing using Tukey's multiple comparison post analysis using GraphPad Prism 6.0 software.

FIG. 3A is a graph of examining a HepG2 cell line as cancer cells derived from human liver. FIG. 3B is a graph of examining an A549 cell line as cancer cells derived from human lung. FIG. 3C is a graph of examining an AGS cell line as cancer cells derived from human stomach. FIG. 3D is a graph of examining a PANC-1 cell line as cancer cells derived from human pancreas. FIG. 3E is a graph of examining a DLD-1 cell line as cancer cells derived from human colon. FIG. 3F is a graph of examining a HeLa cell line as cancer cells derived from human cervix. FIG. 4A is a graph of examining an MDA-MB-231 cell line as cancer cells derived from human breast. FIG. 4B is a graph of examining a PC-3 cell line as cancer cells derived from human prostate. FIG. 4C is a graph of examining an SK-OV-3 cell line as cancer cells derived from human ovary. FIG. 4D is a graph of examining a T24 cell line as cancer cells derived from human bladder. FIG. 4E is a graph of examining a U-87 MG cell line as cancer cells derived from human brain. FIG. 4F is a graph of examining a Saos-2 cell line as cancer cells derived from human bones. ****p<0.0001.

FIG. 5 is a graph showing cell survival rate by MTT assay as a percentage after 48 hours by treating single and complex formulations of MET, 2DG, and IP6 to human-derived normal cell lines at a low concentration usable in human plasma. ****p<0.0001.

FIG. 6 is a diagram illustrated cell survival rate and protein expression in 4T1 cells. A vertical bar of each bar represents a standard deviation. Statistical analysis was performed by one-way ANOVA testing using Tukey's multiple comparison post analysis using GraphPad Prism 6.0 software. FIG. 6A is a graph showing a percentage of cell survival rate by MTT assay after 48 hours of treating MET, 2DG and IP6 alone and in combination on mouse-derived breast cancer 4T1 cells. FIGS. 6B and 6C are diagrams illustrating phosphorylation expression of AMPK and ACC according to single and combination treatment of MET, 2DG and IP6. *p<0.05. ****p<0.0001.

FIG. 7 is a graph of ATP synthesis inhibition of single and complex formulations of MET, 2DG and IP6 for 4T1 cells. Statistical analysis was performed by two-way ANOVA testing using Tukey's multiple comparison post analysis using GraphPad Prism 6.0 software. *p<0.05. ****p<0.0001.

FIG. 8 is a graph of tumor volumes measured at three-day intervals according to administration of single and complex formulations of an anti-PD-1 antibody, MET, 2DG, IP6 and Ins in a test animal. Statistical analysis was performed by two-way ANOVA testing using Tukey's multiple comparison post analysis using GraphPad Prism 6.0 software. **p<0.01, ****p<0.0001.

FIG. 9 is a graph of tumor volumes measured at three-day intervals according to administration of single and complex formulations of an anti-PD-L1 antibody, MET, 2DG, IP6 and Ins in a test animal. Statistical analysis was performed by two-way ANOVA testing using Tukey's multiple comparison post analysis using GraphPad Prism 6.0 software. **p<0.01, ****p<0.0001.

FIG. 10 is a graph of tumor volumes measured at three-day intervals according to administration of single and complex formulations of an anti-CTLA-4 antibody, MET, 2DG, IP6 and Ins in a test animal. Statistical analysis was performed by two-way ANOVA testing using Tukey's multiple comparison post analysis using GraphPad Prism 6.0 software. ***p<0.001, ****p<0.0001.

### MODE FOR INVENTION

Advantages and features of the present invention, and methods for accomplishing the same will be apparent with reference to the embodiments described below in detail. However, the present invention is not limited to the following exemplary embodiments but may be implemented in various different forms. The exemplary embodiments are provided only to complete disclosure of the present invention and to fully provide a person having ordinary skill in the art to which the present invention pertains with the category of the invention, and the present invention will be defined only by the appended claims.

In Examples below, monoclonal antibodies, anti-mouse PD-1 mAb (RMP1-14), anti-mouse PD-L1 mAb (10F. 9G2), anti-mouse CTLA-4 mAb (UC10-4F10-11) were used as an immune checkpoint inhibitor. Further, metformin hydrochloride (Metformin HCl) and phenformin hydrochloride (Phenformin HCl) were used among various pharmaceutically acceptable salt forms of metformin or phenformin as a biguanide-based compound. The form of these salts is not limited by Examples. Further, inositol hexaphosphate (phytic acid) was used among several pharmaceutically acceptable salt forms of inositol hexaphosphate. The form of these salts is not limited by Examples. Further, myo-inositol was used as an isomer of inositol. These isomers are not limited by Examples.

Cell Culture

Cells used in the test were liver cancer (HepG2), lung cancer (A549), stomach cancer (AGS), pancreatic cancer (PANC-1), colon cancer (DLD-1), cervical cancer (HeLa), breast cancer (MDA-MB-231), prostate cancer (PC-3), ovarian cancer (SK-OV-3), bladder cancer (T24), glioblastoma (U-87 MG), osteosarcoma (Saos-2), and mouse-derived breast cancer (4T1) as tumor cells, and prostate (PZ-HPV-7), colon (CCD-18Co), and lung (MRCS) cell lines as non-tumor cells. All cell lines were purchased and used from the Korean Cell Line Bank or US American Type Culture Collection (ATCC) (Rockville, Md.).

The cells were cultured and maintained in a 37° C. incubator (5% CO2/95% air) using a cell culture solution obtained by adding 10% fetal bovine serum (FBS, Hyclone) and 1% penicillin/streptomycin (P/S, Hyclone) to a Roswell Park Memorial Institute 1640 medium (RPMI1640, Hyclone, Logan, Utah, USA). When the cells were filled to about 80% of a culture dish, a single layer of the cells was washed with a phosphate-buffered saline (PBS, Hyclone) and subcultured with 0.25% trypsin-2.65 mM EDTA (Hyclone), and the medium was changed every two days.

Drugs Used

Anti-mouse PD-1 mAb (RMP1-14), anti-mouse PD-L1 mAb (10F.9G2), and anti-mouse CTLA-4 mAb (UC10-4F10-11) as used monoclonal antibodies were purchased from BioXcell and then stored and handled as provided by the manufacturer.

Metformin HCl (hereinafter, referred to as MET), phenformin HCl (hereinafter, referred to as PHE), 2-deoxy-D-glucose (hereinafter, referred to as 2DG), inositol hexaphosphate (phytic acid, hereinafter, referred to as IP6), and myo-inositol (hereinafter, referred to as Ins) were purchased from Sigma (St. Louis, USA). In the present invention, all the drugs used in Table and the drawings summarizing the results obtained through a test were indicated as abbreviations.

### Reference Example 1: Cell Growth Inhibition Assay In Vitro

Cytotoxicity of MET or PHE, 2DG and IP6 was confirmed by MTT assay [3-(4,5-dimethyl thiazolyl-2)-2,5-diphenyltetrazolium bromide assay]. After dispensing the cells (3 to 4×10⁵ cells/well) into a 96-well culture plate and stabilizing for 12 hours or more, the medium of each well was removed and MET or PHE, 2DG, and IP6 for each cell were mixed for each concentration and treated with a medium without serum. For control cells, PBS was added in the medium. After incubation at 37° C. with CO2 for 48 hours, the medium containing the control and the mixture was clearly removed and cultured at 37° C. for 4 hours with an MTT (Sigma Aldrich, St. Louis, Mo., USA) reagent (0.5 mg/ml). Thereafter, the medium containing the MTT reagent was clearly removed and MTT formazan crystals formed by the living cells were left and dissolved at room temperature for 15 minutes or longer by adding DMSO (Sigma). Absorbance was measured at a wavelength of 560 nm using a micro plate reader (BioTek® Instruments, Inc., Winooski, Vt., USA).

### Reference Example 2: Test Method for Single Formulation and Complex Formulation

The cells (3 to 4×10⁵ cells/well) were seeded in a 96-well plate and treated with each of MET or PHE, 2DG, and IP6 as a single formulation for each concentration to confirm a cell proliferation inhibition rate.

The complex formulation drug was treated with a concentration of a drug corresponding to IC50 of a complex formulation consisting of two or more compounds selected from the group consisting of MET or PHE, 2DG and IP6. All cell lines were cultured for 48 hours at the concentration of a single or complex formulation, and a growth inhibition effect was measured by MTT assay.

### Reference Example 3: Test Animals

Five-week-old, female specific pathogen free BALB/c nude mice were purchased and used from DooYeol Biotech Co., Ltd. After quarantine and adaptation for one week, healthy animals without weight loss were selected and used in the test.

The test animals were raised in a breeding environment set at a temperature of 23±3° C., a relative humidity of 50±10%, the ventilation number of 10 to 15 times/hour, lighting time of 12 hours (08:00 to 20:00), and illuminance of 150 to 300 Lux. During a pre-test period, the test animals were allowed to freely consume solid feed for the test animals (Cargill Agripurina Co., Ltd.) and drinking water.

### Reference Example 4: Tumor Cell Transplantation and Test Substance Administration

After an adaptation period for one week, in BALB/c nude mice, 4T1 cells (1×10⁵ cells/mouse), breast cancer cells, were injected to the left breast adipose tissues of the test animals, and then the tumor tissues were visually observed. When the tumor tissue size of the test animals was about 50 mm³, the test animals were divided into 10 test groups based on a randomized block design. That is, the 9 test groups were classified into a control group, an Ins group (Ins 100 mg/kg), a IP6 group (IP6 1000 mg/kg), a MET group (MET 500 mg/kg), a 2DG group (2DG 1000 mg/kg), a mAb group (150 m/mouse as monoclonal antibody (anti-PD-1 antibody or anti-PD-L1 antibody or anti-CTLA-4 antibody)), a mAb+MET+2DG group (mAb 150 m/mouse+MET 500 mg/kg+2DG 1000 mg/kg), a mAb+MET+2DG+Ins group (mAb 150 m/mouse+MET 500 mg/kg+2DG 1000 mg/kg+Ins 1000 mg/kg), a mAb+MET+2DG+IP6 group (mAb 150 m/mouse+MET 500 mg/kg+2DG 1000 mg/kg+IP6 1000 mg/kg), a mAb+MET+2DG+IP6+Ins group (mAb 150 μg/mouse+MET 500 mg/kg+2DG 1000 mg/kg+IP6 500 mg/kg+Ins 500 mg/kg), and each test group used 10 test animals. A test substance monoclonal antibody was intraperitoneally administered every four days by setting a test group separation time to 1 day, and test substances Ins, IP6, MET, and 2DG were dissolved in distilled water until a test end time by setting a test group separation time to 1 day and orally administered at a predetermined time for three weeks.

### Reference Example 5: Measurement of Body Weight of Test Animal and Tumor Volume

The body weight of the test animal during the test period was measured at a fixed time once a week from the test substance administration date. A tumor volume was measured by using a digital caliper every three days, the length and width of the tumor were measured, and the tumor volume was calculated by substituting the following Equation.

Tumor volume (mm³)=(width²×length)/2

### Reference Example 6: Statistical Processing

All analysis values were expressed as mean±SD. To compare a difference between a control group and a test substance-treated group, significance was verified by one-way ANOVA or two-way ANOVA testing using Tukey's multiple comparison post analysis using GraphPad Prism 6.0 software. It was determined that there was statistical significance only when p<0.05 or more.

### Example 1. Cell Proliferation Inhibition Test of Single Formulation and Complex Formulation of MET (or PHE), 2DG and IP6

Cell proliferation inhibition effects of a single formulation and a complex formulation of MET (or PHE), 2DG and IP6 were compared using 12 types of cancer cells.

### Example 1-1: Cell Survival Rate After Administering MET, 2DG and IP6 Alone and in Combination

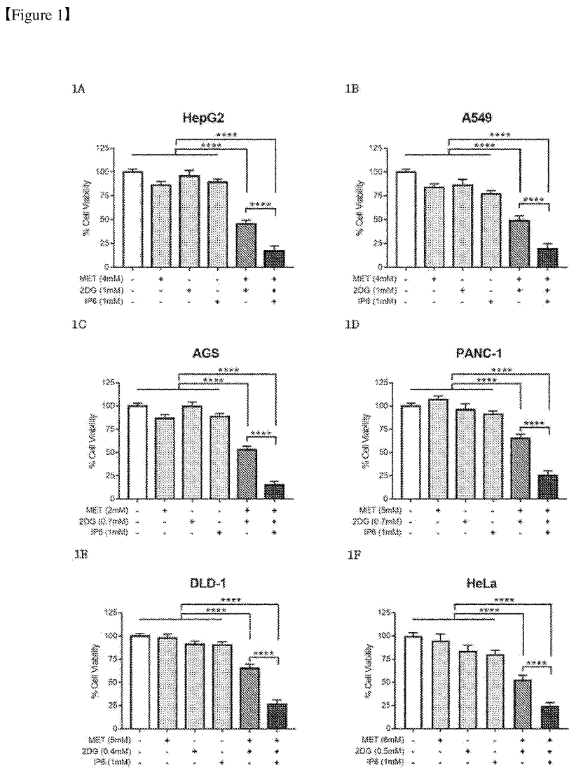

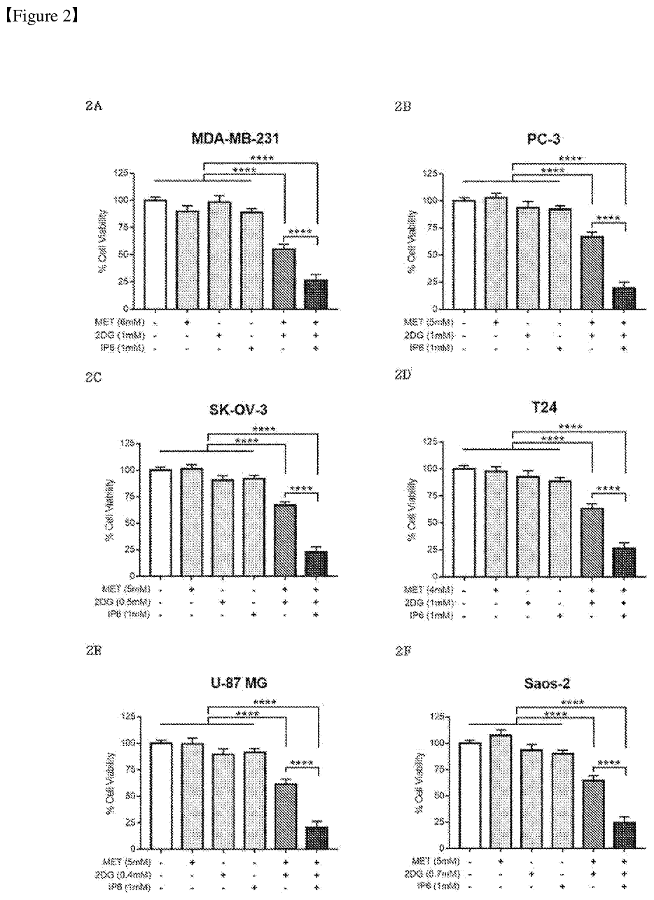

FIGS. 1 and 2 are diagrams of examining cell survival rate after administering MET, 2DG and IP6 alone or in combination based on human cancer cell lines such as liver cancer (HepG2), lung cancer (A549), gastric cancer (AGS), pancreatic cancer (PANC-1), colon cancer (DLD-1), cervical cancer (HeLa), breast cancer (MDA-MB-231), prostate cancer (PC-3), ovarian cancer (SK-OV-3), bladder cancer (T24), glioblastoma (U-87 MG), and osteosarcoma (Saos-2).

FIG. 1A shows cell survival rate in a liver cancer (HepG2) cell line treated alone and in combination with 4 mM of MET, 1 mM of 2DG, and 1 mM of IP6. A combination-treated formulation had significantly lower survival rate than single treatment including a control (P<0.0001). Compared between the combination-treated groups, the cell survival rate of a combination of MET+2DG+IP6 was 17.44±4.8%, which was 2.6 times significantly lower than 45.40±4.2%, the cell survival rate of a combination of MET+2DG (P<0.0001).

FIG. 1B shows cell survival rate in a lung cancer (A549) cell line treated alone and in combination with 4 mM of MET, 1 mM of 2DG, and 1 mM of IP6. A combination-treated formulation had significantly lower survival rate than single treatment including a control (P<0.0001). Compared between the combination-treated groups, the cell survival rate of a combination of MET+2DG+IP6 was 19.43±5.2%, which was 2.5 times significantly lower 48.84±5.3%, the cell survival rate of a combination of MET+2DG (P<0.0001).

FIG. 1C shows cell survival rate in a stomach cancer (AGS) cell line treated alone and in combination with 2 mM of MET, 0.7 mM of 2DG, and 1 mM of IP6. A combination-treated formulation had significantly lower survival rate than single treatment including a control (P<0.0001). Compared between the combination-treated groups, the cell survival rate of a combination of MET+2DG+IP6 was 15.20±4.2%, which was 3.5 times significantly lower than 53.00±3.8%, the cell survival rate of a combination of MET+2DG (P<0.0001).

FIG. 1D shows cell survival rate in a pancreatic cancer (PANC-1) cell line treated alone and in combination with 5 mM of MET, 0.7 mM of 2DG, and 1 mM of IP6. A combination-treated formulation had significantly lower survival rate than single treatment including a control (P<0.0001). Compared between the combination-treated groups, the cell survival rate of a combination of MET+2DG+IP6 was 25.27±5.2%, which was 2.6 times significantly lower than 65.40±4.3%, the cell survival rate of a combination of MET+2DG (P<0.0001).

FIG. 1E shows cell survival rate in a colon cancer (DLD-1) cell line treated alone and in combination with 5 mM of MET, 0.4 mM of 2DG, and 1 mM of IP6. A combination-treated formulation had significantly lower survival rate than single treatment including a control (P<0.0001). Compared between the combination-treated groups, the cell survival rate of a combination of MET+2DG+IP6 was 26.70±4.7%, which was 2.4 times significantly lower than 65.40±4.6%, the cell survival rate of a combination of MET+2DG (P<0.0001).

FIG. 1F shows cell survival rate in a cervical cancer (HeLa) cell line treated alone and in combination with 6 mM of MET, 0.5 mM of 2DG, and 1 mM of IP6. A combination-treated formulation had significantly lower survival rate than single treatment including a control (P<0.0001). Compared between the combination-treated groups, the cell survival rate of a combination of MET+2DG+IP6 was 24.67±3.6%, which was 2.1 times significantly lower than 52.89±4.6%, the cell survival rate of a combination of MET+2DG (P<0.0001).

FIG. 2A shows cell survival rate in a breast cancer (MDA-MB-231) cell line treated alone and in combination with 6 mM of MET, 1 mM of 2DG, and 1 mM of IP6. A combination-treated formulation had significantly lower survival rate than single treatment including a control (P<0.0001). Compared between the combination-treated groups, the cell survival rate of a combination of MET+2DG+IP6 was 26.37±5.3%, which was 2.1 times significantly lower than 55.15±4.5%, the cell survival rate of a combination of MET+2DG (P<0.0001).

FIG. 2B shows cell survival rate in a prostate cancer (PC-3) cell line treated alone and in combination with 5 mM of MET, 1 mM of 2DG, and 1 mM of IP6. A combination-treated formulation had significantly lower survival rate than single treatment including a control (P<0.0001). Compared between the combination-treated groups, the cell survival rate of a combination of MET+2DG+IP6 was 19.51±5.6%, which was 3.4 times significantly lower than 66.70±4.6%, the cell survival rate of a combination of MET+2DG (P<0.0001).

FIG. 2C shows cell survival rate in an ovarian cancer (SK-OV-3) cell line treated alone and in combination with 5 mM of MET, 0.5 mM of 2DG, and 1 mM of IP6. A combination-treated formulation had significantly lower survival rate than single treatment including a control (P<0.0001). Compared between the combination-treated groups, the cell survival rate of a combination of MET+2DG+IP6 was 22.80±5.2%, which was 2.9 times significantly lower than 66.80±3.6%, the cell survival rate of a combination of MET+2DG (P<0.0001).

FIG. 2D shows cell survival rate in a bladder cancer (T24) cell line treated alone and in combination with 4 mM of MET, 1 mM of 2DG, and 1 mM of IP6. A combination-treated formulation had significantly lower survival rate than single treatment including a control (P<0.0001). Compared between the combination-treated groups, the cell survival rate of a combination of MET+2DG+IP6 was 26.42±4.8%, which was 2.4 times significantly lower than 63.30±4.2%, the cell survival rate of a combination of MET+2DG (P<0.0001).

FIG. 2E shows cell survival rate in a glioblastoma (U-87 MG) cell line treated alone and in combination with 5 mM of MET, 0.4 mM of 2DG, and 1 mM of IP6. A combination-treated formulation had significantly lower survival rate than single treatment including a control (P<0.0001). Compared between the combination-treated groups, the cell survival rate of a combination of MET+2DG+IP6 was 20.80±5.7%, which was 2.9 times significantly lower than 61.32±4.8%, the cell survival rate of a combination of MET+2DG (P<0.0001).

FIG. 2F shows cell survival rate in an osteosarcoma (Saos-2) cell line treated alone and in combination with 5 mM of MET, 0.7 mM of 2DG, and 1 mM of IP6. A combination-treated formulation had significantly lower survival rate than single treatment including a control (P<0.0001). Compared between the combination-treated groups, the cell survival rate of a combination of MET+2DG+IP6 was 24.52±5.7%, which was 2.6 times significantly lower than 64.33±4.9%, the cell survival rate of a combination of MET+2DG (P<0.0001).

From these results, it was confirmed that the triple complex formulation of MET, 2DG and IP6 was a better cancer cell proliferation inhibitory effect than the single or double complex formulation.

### Example 1-2: Cell Survival Rate after Administering Phe, 2Dg and Ip6 Alone and in Combination

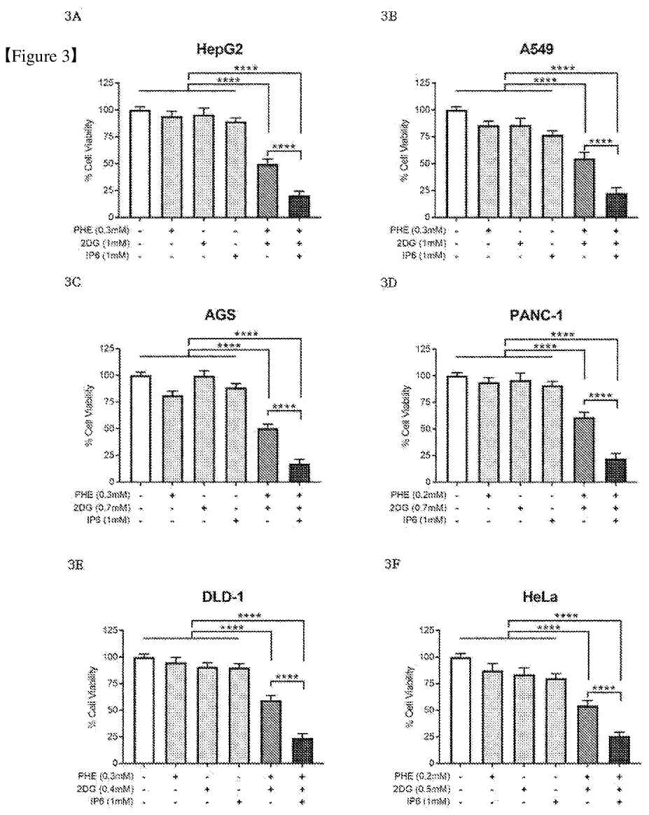

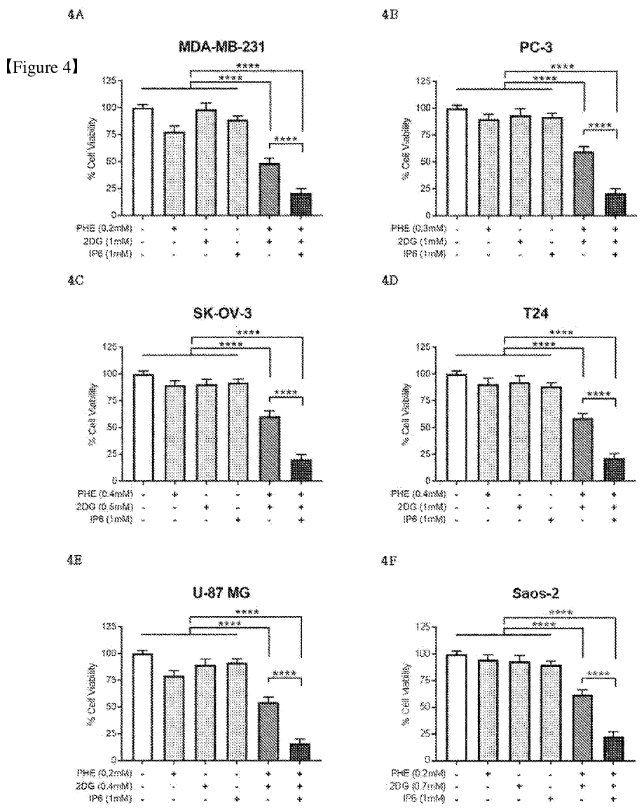

FIGS. 3 and 4 are diagrams of examining cell survival rate after administering PHE, 2DG and IP6 alone or in combination based on human cancer cell lines such as liver cancer (HepG2), lung cancer (A549), gastric cancer (AGS), pancreatic cancer (PANC-1), colon cancer (DLD-1), cervical cancer (HeLa), breast cancer (MDA-MB-231), prostate cancer (PC-3), ovarian cancer (SK-OV-3), bladder cancer (T24), glioblastoma (U-87 MG), and osteosarcoma (Saos-2).

FIG. 3A shows cell survival rate in a liver cancer (HepG2) cell line treated alone and in combination with 0.3 mM of PHE, 1 mM of 2DG, and 1 mM of IP6. A combination-treated formulation had significantly lower survival rate than single treatment including a control (P<0.0001). Compared between the combination-treated groups, the cell survival rate of a combination of PHE+2DG+IP6 was 20.21±4.1%, which was 2.5 times significantly lower than 49.55±4.8%, the cell survival rate of a combination of PHE+2DG (P<0.0001).

FIG. 3B shows cell survival rate in a lung cancer (A549) cell line treated alone and in combination with 0.3 mM of PHE, 1 mM of 2DG, and 1 mM of IP6. A combination-treated formulation had significantly lower survival rate than single treatment including a control (P<0.0001). Compared between the combination-treated groups, the cell survival rate of a combination of PHE+2DG+IP6 was 22.41±5.2%, which was 2.4 times significantly lower than 54.77±5.8%, the cell survival rate of a combination of PHE+2DG (P<0.0001).

FIG. 3C shows cell survival rate in a stomach cancer (AGS) cell line treated alone and in combination with 0.3 mM of PHE, 0.7 mM of 2DG, and 1 mM of IP6. A combination-treated formulation had significantly lower survival rate than single treatment including a control (P<0.0001). Compared between the combination-treated groups, the cell survival rate of a combination of PHE+2DG+IP6 was 17.38±3.8%, which was 2.9 times significantly lower than 50.45±3.9%, the cell survival rate of a combination of PHE+2DG (P<0.0001).

FIG. 3D shows cell survival rate in a pancreatic cancer (PANC-1) cell line treated alone and in combination with 0.2 mM of PHE, 0.7 mM of 2DG, and 1 mM of IP6. A combination-treated formulation had significantly lower survival rate than single treatment including a control (P<0.0001). Compared between the combination-treated groups, the cell survival rate of a combination of PHE+2DG+IP6 was 25.27±5.2%, which was 2.6 times significantly lower than 65.40±4.3%, the cell survival rate of a combination of PHE+2DG (P<0.0001).

FIG. 3E shows cell survival rate in a colon cancer (DLD-1) cell line treated alone and in combination with 0.3 mM of PHE, 0.4 mM of 2DG, and 1 mM of IP6. A combination-treated formulation had significantly lower survival rate than single treatment including a control (P<0.0001). Compared between the combination-treated groups, the cell survival rate of a combination of PHE+2DG+IP6 was 22.21±4.8%, which was 2.7 times significantly lower than 60.98±4.7%, the cell survival rate of a combination of PHE+2DG (P<0.0001).

FIG. 3F shows cell survival rate in a cervical cancer (HeLa) cell line treated alone and in combination with 0.2 mM of PHE, 0.5 mM of 2DG, and 1 mM of IP6. A combination-treated formulation had significantly lower survival rate than single treatment including a control (P<0.0001). Compared between the combination-treated groups, the cell survival rate of a combination of PHE+2DG+IP6 was 25.65±3.9%, which was 2.1 times significantly lower than 54.67±4.6%, the cell survival rate of a combination of PHE+2DG (P<0.0001).

FIG. 4A shows cell survival rate in a breast cancer (MDA-MB-231) cell line treated alone and in combination with 0.2 mM of PHE, 1 mM of 2DG, and 1 mM of IP6. A combination-treated formulation had significantly lower survival rate than single treatment including a control (P<0.0001). Compared between the combination-treated groups, the cell survival rate of a combination of PHE+2DG+IP6 was 20.76±4.2%, which was 2.3 times significantly lower than 48.54±4.5%, the cell survival rate of a combination of PHE+2DG (P<0.0001).

FIG. 4B shows cell survival rate in a prostate cancer (PC-3) cell line treated alone and in combination with 0.3 mM of PHE, 1 mM of 2DG, and 1 mM of IP6. A combination-treated formulation had significantly lower survival rate than single treatment including a control (P<0.0001). Compared between the combination-treated groups, the cell survival rate of a combination of PHE+2DG+IP6 was 20.77±4.3%, which was 2.9 times significantly lower than 59.66±4.6%, the cell survival rate of a combination of PHE+2DG (P<0.0001).

FIG. 4C shows cell survival rate in an ovarian cancer (SK-OV-3) cell line treated alone and in combination with 0.4 mM of PHE, 0.5 mM of 2DG, and 1 mM of IP6. A combination-treated formulation had significantly lower survival rate than single treatment including a control (P<0.0001). Compared between the combination-treated groups, the cell survival rate of a combination of PHE+2DG+IP6 was 20.44±4.2%, which was 3.0 times significantly lower than 60.54±5.1%, the cell survival rate of a combination of PHE+2DG (P<0.0001).

FIG. 4D shows cell survival rate in a bladder cancer (T24) cell line treated alone and in combination with 0.4 mM of PHE, 1 mM of 2DG, and 1 mM of IP6. A combination-treated formulation had significantly lower survival rate than single treatment including a control (P<0.0001). Compared between the combination-treated groups, the cell survival rate of a combination of PHE+2DG+IP6 was 21.45±4.2%, which was 2.7 times significantly lower than 58.70±4.5%, the cell survival rate of a combination of PHE+2DG (P<0.0001).

FIG. 4E shows cell survival rate in a glioblastoma (U-87 MG) cell line treated alone and in combination with 0.2 mM of PHE, 0.4 mM of 2DG, and 1 mM of IP6. A combination-treated formulation had significantly lower survival rate than single treatment including a control (P<0.0001). Compared between the combination-treated groups, the cell survival rate of a combination of PHE+2DG+IP6 was 15.76±4.2%, which was 3.4 times significantly lower than 54.32±4.8%, the cell survival rate of a combination of PHE+2DG (P<0.0001).

FIG. 4F shows cell survival rate in an osteosarcoma (Saos-2) cell line treated alone and in combination with 0.2 mM of PHE, 0.7 mM of 2DG, and 1 mM of IP6. A combination-treated formulation had significantly lower survival rate than single treatment including a control (P<0.0001). Compared between the combination-treated groups, the cell survival rate of a combination of PHE+2DG+IP6 was 22.76±4.2%, which was 2.7 times significantly lower than 61.93±4.7%, the cell survival rate of a combination of PHE+2DG (P<0.0001).

From these results, it was confirmed that the triple-complex formulation of PHE, 2DG and IP6 was a better cancer cell proliferation inhibitory effect than the single or double-complex formulation.

### Example 2: Effect of Complex Formulation of MET, 2DG and IP6 on Normal Cells

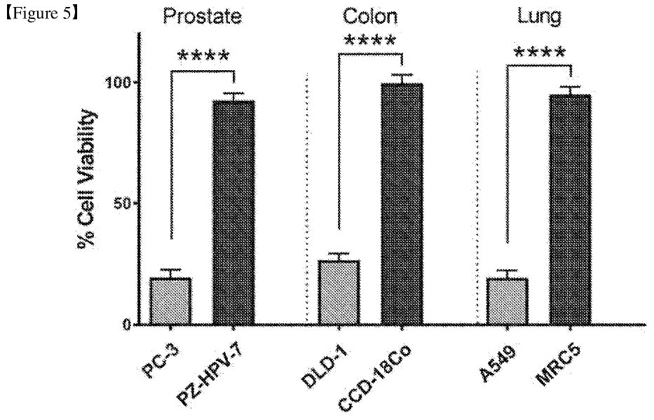

FIG. 5 is a diagram of examining cytotoxicity by MTT assay after 48 hours after treating a three-combined formulation to prostate cancer (PC-3), colon cancer (DLD-1), and lung cancer (A549) cell lines as tumor cells and prostate (PZ-HPV-7), colon (CCD-18Co) and lung (MRCS) cell lines as non-tumor cells in order to look into an effect of a complex formulation of MET, 2DG and IP6 on normal cells.

As a result in PC-3 and PZ-HPV-7 cell lines treated with a complex formulation of 5 mM of MET, 1 mM of 2DG, and 1 mM of IP6, the cell survival rate was significantly reduced in a PC-3 cell line as tumor cells, while a PZ-HPV-7 cell line as non-tumor cells did not affect cell survival rate (P<0.0001).

As a result in DLD-1 and CCD-18Co cell lines treated with a complex formulation of 5 mM of MET, 0.4 mM of 2DG, and 1 mM of IP6, the cell survival was significantly reduced in a DLD-1 cell line as tumor cells, while a CCD-18Co cell line as non-tumor cells did not affect cell survival rate (P<0.0001).

As a result in A549 and MRCS cell lines treated with a complex formulation of 4 mM of MET, 1 mM of 2DG, and 1 mM of IP6, the cell survival rate was significantly reduced in a A549 cell line as tumor cells, while a MRCS cell line as non-tumor cells did not affect cell survival rate (P<0.0001).

Apoptosis for each non-tumor cells of the three-combined formulation showed a different pattern from tumor cells and the three-combined formulation was confirmed to be a safe drug in vivo.

### Example 3: Cell Survival Rate and Protein Expression of 4T1 Cells by Single and Complex Formulations of MET, 2DG and IP6

The metabolism of living cells uses ATP and ADP as energy sources and produces AMPs. AMP-activated protein kinase (AMPK) is a serine/threonine kinase known as a regulator of lipid and glucose metabolism and plays an important regulatory role in ophthalmic diabetes. The AMPK is activated by AMP to inhibit ATP use, wherein AMP increases when cellular energy is consumed, and plays a key role in maintaining homeostasis by inducing catabolism. AMPK activation inhibits the proliferation of cancer cells and inhibits acetyl CoA carboxylase (ACC), an enzyme that induces fatty acid synthesis in terms of fat metabolism.

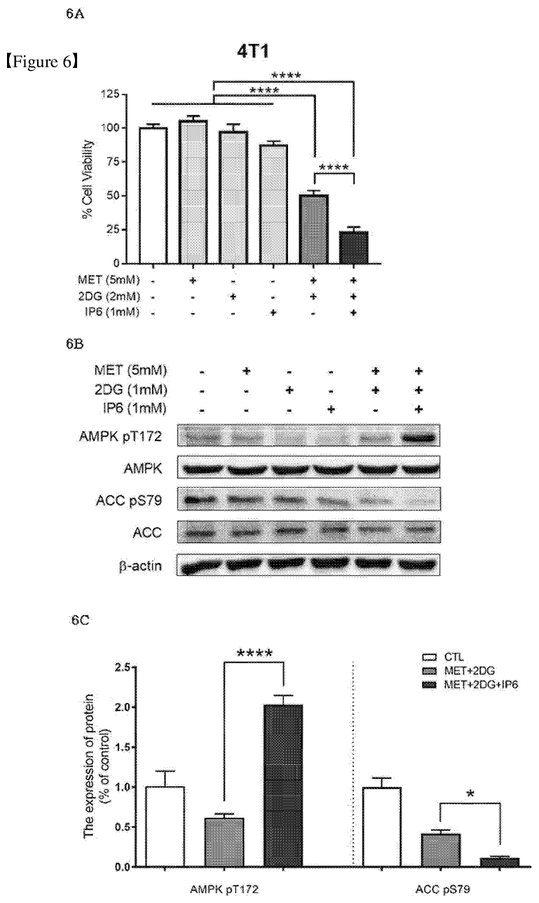

FIG. 6A shows cell survival rate in a mouse-derived breast cancer (4T1) cell line treated alone and in combination with 5 mM of MET, 2 mM of 2DG, and 1 mM of IP6. A combination-treated formulation had significantly lower survival rate than single treatment including a control (P<0.0001). In comparison between the combination-treated groups, the cell survival rate of a (MET+2DG+IP6) group was 23.04±4.0%, which was reduced 2.2 times significantly lower than 50.03±4.0%, the cell survival rate of a (MET+2DG) group (P<0.0001).

In FIGS. 6B and 6C, AMPK is significantly activated (P<0.0001) and phosphorylation of ACC is reduced (P<0.05) in the (MET+2DG+IP6) group compared with the (MET+2DG) group.

### Example 4. ATP Synthesis Inhibition of Single and Complex Formulations of MET, 2DG and IP6 for 4T1 Cells

ATP (adenosine triphosphate) is an energy source for living organisms, and when intracellular ATP synthesis is inhibited, energy metabolism activity is reduced. An ATP synthesis inhibitory effect of MET, 2DG, and IP6 was confirmed in a mouse-derived breast cancer cell line 4T1 of Example 3.

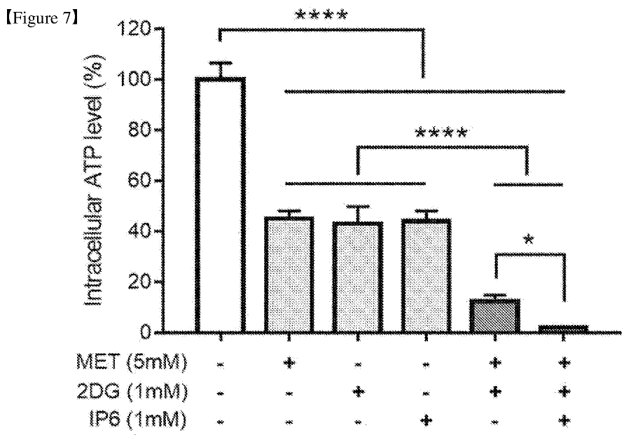

Each cell (10³-10⁴ cells) of 4T1 was incubated for 24 hours in a 60 mm culture dish, and treated alone and in combination with 4 mM of MET, 1 mM of 2DG, and 1 mM of IP6, and further incubated for 48 hours. Thereafter, cells were harvested and counted and diluted in 100 ml of an RPMI culture solution containing 10 volume % FBS, and was transferred to each well of a 96-well plate. 100 ml of an assay buffer (rL/L reagent+reconstitution buffer) of a Promega ATP assay kit (G7572, Promega, Durham, N.C., USA) was added to the wells containing the cells, and the emission of fluorescence was measured at 560 nm. The results were shown in FIG. 7.

As shown in FIG. 7, test results showed that ATP synthesis was inhibited in the combination treatment rather than single treatment in single and combination treatments in a 4T1 cell line used in the test (P<0.0001). The MET+2DG+IP6 group was found to significantly inhibit ATP synthesis as compared with the MET+2DG group (P<0.05). As a result, it can be seen that the complex formulation of the MET+2DG+IP6 group reduces the energy level most effectively in cancer cells. In Examples 5 to 7 below, a tumor growth inhibition effect was shown by administering the test substances Ins, IP6, MET, 2DG in combination with an immune checkpoint inhibitor.

### Example 5. Effect of Single and Complex Formulations of Anti-PD-1, Ins, IP6, MET and 2DG on Tumor Volume Change

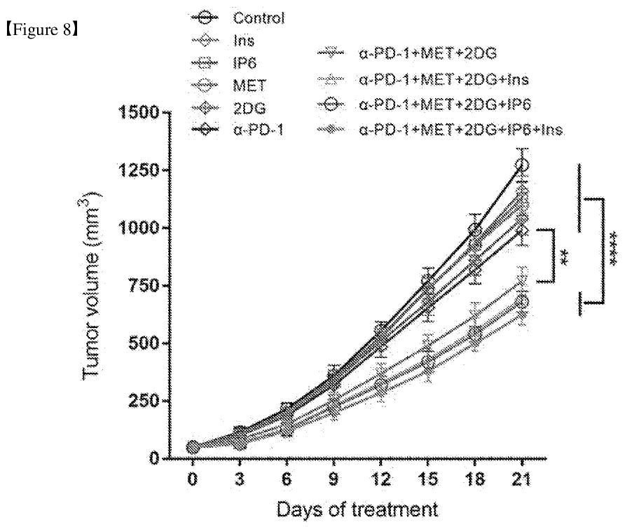

When the size of a tumor tissue was about 50 mm³, test groups were classified and test substance administration was started, and the tumor volume was measured at 3 days intervals from the start date of the test substance administration. As a result of the test, as shown in FIG. 8, the volumes of tumors of single and combination-treated groups were reduced compared to a control group from the day 3 of the test substance administration. On day 21 of test substance administration, an anti-PD-1 group administered with only a monoclonal antibody was significantly different from an anti-PD-1+MET+2DG group (P<0.01). In addition, a control group, an Ins group, an IP6 group, a MET group, a 2DG group, and an anti-PD-1 group had a significant difference higher than an anti-PD-1+MET+2DG+Ins group, an anti-PD-1+MET+2DG+IP6 group, and an anti-PD-1+MET+2DG+IP6+Ins group which were four or more complex formulations containing monoclonal antibodies (P<0.0001). Among total 10 groups, the anti-PD-1+MET+2DG+IP6+Ins group showed the highest decrease in tumor volume and exhibited a high tumor growth inhibitory effect (FIG. 8).

### Example 6. Effect of Single and Complex Formulations of Anti-PD-L1, Ins, IP6, MET and 2DG on Tumor Volume Change

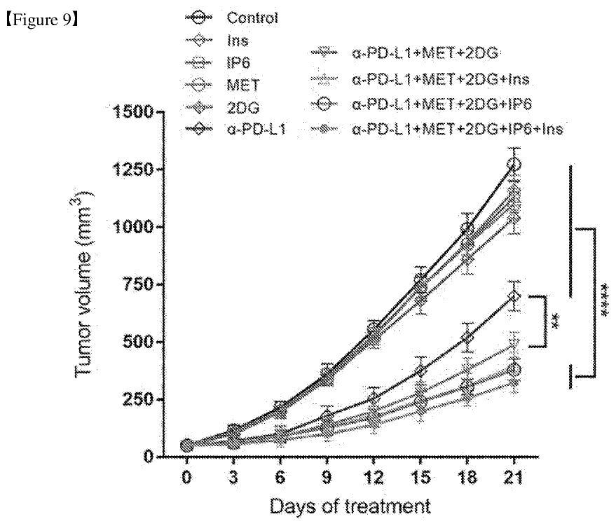

When the size of tumor tissue was about 50 mm³, test groups were classified and test substance administration was started, and the tumor volume was measured at 3 days intervals from the start of the test substance administration. As a result of the test, as shown in FIG. 9, the volumes of tumors of single and combination-treated groups were reduced compared to a control group from the day 3 of the test substance administration. On day 21 of test substance administration, an anti-PD-L1 group administered with only a monoclonal antibody was significantly different from an anti-PD-L1+MET+2DG group (P<0.01). In addition, a control group, an Ins group, an IP6 group, a MET group, a 2DG group, and an anti-PD-L1 group had a significant difference higher than an anti-PD-L1+MET+2DG+Ins group, an anti-PD-L1+MET+2DG+IP6 group, and an anti-PD-L1+MET+2DG+IP6+Ins group which were four or more complex formulations containing monoclonal antibodies (P<0.0001). Among total 10 groups, the anti-PD-L1+MET+2DG+IP6+Ins group showed the highest decrease in tumor volume and exhibited a high tumor growth inhibitory effect (FIG. 9).

### Example 7. Effect of Single and Complex Formulations of Anti-Ctla-4, Ins, Ip6, MET and 2DG on Tumor Volume Change

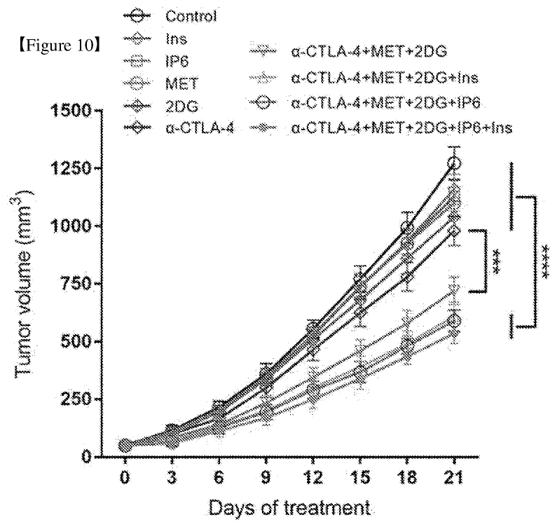

When the size of tumor tissue was about 50 mm³, test groups were classified and test substance administration was started, and the tumor volume was measured at 3 days intervals from the start of the test substance administration. As a result of the test, as shown in FIG. 10, the volumes of tumors of single and combination-treated groups were reduced compared to a control group from the day 3 of the experimental substance administration. On day 21 of test substance administration, an anti-CTLA-4 group administered with only a monoclonal antibody was significantly different from an anti-CTLA-4+MET+2DG group (P<0.001). In addition, a control group, an Ins group, an IP6 group, a MET group, a 2DG group, and an anti-CTLA-4 group had a significant difference higher than an anti-CTLA-4+MET+2DG+Ins group, an anti-CTLA-4+MET+2DG+IP6 group, and an anti-CTLA-4+MET+2DG+IP6+Ins group which were four or more complex formulations containing monoclonal antibodies (P<0.0001). Among total 10 groups, the anti-CTLA-4+MET+2DG+IP6+Ins group showed the highest decrease in tumor volume and exhibited a high tumor growth inhibitory effect (FIG. 10).

1.-19. (canceled)

20. A method for treating cancer comprising administering a therapeutically effective amount of:
an immune checkpoint inhibitor;
a biguanide-based compound or a pharmaceutically acceptable salt thereof; and
2-deoxy-D-glucose to a subject in need of treatment thereof.

21. The method of claim 20, further administering a therapeutically effective amount of inositol hexaphosphate or a pharmaceutically acceptable salt thereof, inositol, or a mixture thereof.

22. The method of claim 21, wherein the administering comprises administering a therapeutically effective amount of
an immune checkpoint inhibitor;
a biguanide-based compound or a pharmaceutically acceptable salt thereof;
2-deoxy-D-glucose; and
inositol hexaphosphate or a pharmaceutically acceptable salt thereof.

23. The method of claim 21, wherein the administering comprises administering a therapeutically effective amount of
an immune checkpoint inhibitor;
a biguanide-based compound or a pharmaceutically acceptable salt thereof;
2-deoxy-D-glucose; and
inositol.

24. The method of claim 21, wherein the administering comprises administering a therapeutically effective amount of
an immune checkpoint inhibitor;
a biguanide-based compound or a pharmaceutically acceptable salt thereof;
2-deoxy-D-glucose; and
inositol hexaphosphate and inositol.

25. The method of claim 20, wherein the immune checkpoint inhibitor comprises at least one selected from the group of an anti-CTLA4 antibody, an anti-PD-1 antibody, an anti-LAG-3 antibody, an anti-OX40 antibody, an anti-TIM3 antibody, and an anti-PD-L1 antibody.

26. The method of claim 25, wherein the immune checkpoint inhibitor comprises at least one selected from the group of an anti-CTLA4 antibody, an anti-PD-1 antibody, and an anti-PD-L1 antibody.

27. The method of claim 26, wherein a single dosage of the immune checkpoint inhibitor is in a range of 0.01 to 25 mg/kg.

28. The method of claim 20, wherein the biguanide-based compound comprises at least one selected from metformin and phenformin.

29. The method of claim 21, wherein the inositol compound comprises at least one selected from the group of D-chiro-inositol, L-chiro-inositol, myo-inositol, and scyllo-inositol.

30. The method of claim 20, wherein the cancer is at least one selected from the group consisting of liver cancer, lung cancer, stomach cancer, pancreatic cancer, colon cancer, cervical cancer, breast cancer, prostate cancer, ovarian cancer, brain cancer, osteosarcoma, bladder cancer, head and neck cancer, kidney cancer, melanoma, leukemia, and lymphoma.

31. The method of claim 20, wherein the immune checkpoint inhibitor comprises at least one selected from the group of an anti-CTLA4 antibody, an anti-PD-1 antibody, and an anti-PD-L1 antibody; and
wherein a biguanide-based compound or a pharmaceutically acceptable salt thereof comprises at least one selected from metformin and phenformin.

32. The method of claim 21, wherein the administering comprises administering a therapeutically effective amount of
an immune checkpoint inhibitor;
a biguanide-based compound or a pharmaceutically acceptable salt thereof;
2-deoxy-D-glucose; and
inositol hexaphosphate or a pharmaceutically acceptable salt thereof;
wherein the immune checkpoint inhibitor comprises at least one selected from the group of an anti-CTLA4 antibody, an anti-PD-1 antibody, and an anti-PD-L1 antibody; and
wherein a biguanide-based compound or a pharmaceutically acceptable salt thereof comprises at least one selected from metformin and phenformin.

33. The method of claim 21, wherein the administering comprises administering a therapeutically effective amount of
an immune checkpoint inhibitor;
a biguanide-based compound or a pharmaceutically acceptable salt thereof;
2-deoxy-D-glucose; and
Inositol;
wherein the immune checkpoint inhibitor comprises at least one selected from the group of an anti-CTLA4 antibody, an anti-PD-1 antibody, and an anti-PD-L1 antibody; and
wherein a biguanide-based compound or a pharmaceutically acceptable salt thereof comprises at least one selected from metformin and phenformin.

34. The method of claim 21, wherein the administering comprises administering a therapeutically effective amount of
an immune checkpoint inhibitor;
a biguanide-based compound or a pharmaceutically acceptable salt thereof;
2-deoxy-D-glucose; and
inositol hexaphosphate and inositol;
wherein the immune checkpoint inhibitor comprises at least one selected from the group of an anti-CTLA4 antibody, an anti-PD-1 antibody, and an anti-PD-L1 antibody; and
wherein a biguanide-based compound or a pharmaceutically acceptable salt thereof comprises at least one selected from metformin and phenformin.

35. A combination comprising an immune checkpoint inhibitor; a biguanide-based compound or a pharmaceutically acceptable salt thereof; and 2-deoxy-D-glucose.

36. The combination of claim 35, the combination further comprises inositol hexaphosphate or a pharmaceutically acceptable salt thereof, inositol, or a mixture thereof.

37. The combination of claim 35, wherein the immune checkpoint inhibitor is at least one selected from the group of an anti-CTLA4 antibody, an anti-PD-1 antibody, and an anti-PD-L1 antibody.

38. The combination of claim 35, wherein a biguanide-based compound or a pharmaceutically acceptable salt thereof comprises at least one selected from metformin and phenformin.

39. The combination of claim 35, wherein the immune checkpoint inhibitor is at least one selected from the group of an anti-CTLA4 antibody, an anti-PD-1 antibody, and an anti-PD-L1 antibody; and wherein a biguanide-based compound or a pharmaceutically acceptable salt thereof comprises at least one selected from metformin and phenformin.
